(19) United States
(12) Patent Application Publication
(10) Pub. No.: US 2025/0268252 A1
(43) Pub. Date: Aug. 28, 2025

(54) NANOCARRIER FOR BIOMATERIAL DELIVERY, NANOCARRIER-BIOMATERIAL COMPLEX, MANUFACTURING METHODS THEREOF AND TREATMENT METHOD FOR LIVING PLANT-BASED OBJECT USING NANOCARRIER

(71) Applicant: Seoul National University R&DB foundation, Seoul (KR)

(72) Inventors: Seon Yeong Kwak, Seoul (KR); Pil Joon Seo, Daejeon (KR); Woojin Jeong, Seoul (KR); Yeong Yeop Jeong, Seoul (KR)

(21) Appl. No.: 19/062,772

(22) Filed: Feb. 25, 2025

(30) Foreign Application Priority Data
Feb. 27, 2024 (KR) 10-2024-0027910

(51) Int. Cl. A01N 25/34; A01N 63/60

(52) U.S. Cl. CPC A01N 25/34; A01N 63/60

(57) ABSTRACT

The present disclosure provides a nanocarrier for delivering biomaterials into a living plant-based object including a nanotube, and a functional compound bound to a surface of the nanotubes and including an imidazole, wherein the nanocarrier is configured to deliver a ribonucleic acid (RNA) that promotes a regeneration of plant cells of the object as the biomaterial, into the object.

### CROSS-REFERENCE TO RELATED APPLICATION

This application claims priority of Korean Patent Application No. 10-2024-0027910, filed on Feb. 27, 2024, in the KIPO (Korean Intellectual Property Office), the disclosure of which is incorporated herein entirely by reference.

### BACKGROUND OF THE INVENTION

### Field of the Invention

The present invention relates to a carrier for material delivery, a method of fabricating the same and a utilization of the same, and more specifically, to a nanocarrier, a complex comprising the same and method of fabricating the same.

### Description of the Related Art

Population growth and global warming are driving a need to develop new plant breeding and increase crop yields. Plant regeneration is an essential technology for genetic engineering applications such as genome editing and transformation for crop improvement, and low regeneration efficiency is known to be a major hurdle in genome editing (engineering). For agrobiotechnological breeding of high-value crops, genome editing or transformation of plant cells is followed by tissue de-differentiation (i.e. callus formation) and aerial part redifferentiation (i.e. shoot regeneration). However, the efficiency of the plant redifferentiation process is very low, so the trait improvement in major crops is very limited. With the recent advancement of CRISPR technology, the demand and market for precision breeding has grown explosively, and technologies to improve the efficiency of plant regeneration are essential in this market.

In the past, various hormones and additives have been added to culture medium for a regeneration to improve the regeneration efficiency of plants (crops), but the approach face distinct limitations. The low regeneration efficiency of these plants (crops) has been attributed to genetic barriers or genetic factors. Therefore, controlling gene expression (genetic factors) that inherently act as a barrier to the regeneration during tissue culture may be key to improving regeneration efficiency.

MicroRNAs (hereafter, miRNAs), known as genetic factors with great influence on the regeneration process of plant body, have short-single-stranded RNAs with an average length of 22 nucleotides, and they are expressed in cells and are known to inhibit the expression of target genes by binding complementarily to the nucleic sequence of the messenger RNA (mRNA) of the target gene and inhibiting the translation of the mRNA into protein by the ribosome, or by causing the mRNA to be decomposed.

While information of the various genes involved in the regeneration process of plant has been disclosed, efficient methodologies to control it are very limited. Currently, methods such as transformation using Agrobacterium tumefaciens or gene guns are being utilized to overcome genetic factors, but they are difficult to apply to a wide range of crops due to the limitations of the target crops and genetic modification (GM) issues. Therefore, there is a need to develop gene delivery materials that may temporarily control gene expression only at a specific time without transformation, and utilize them to overcome the limitations of—regeneration of plant callus. In particular, it is necessary to develop new materials that may be applied regardless of plant species and to stably deliver appropriate biomaterials into the callus of various crop species.

### SUMMARY OF THE INVENTION

The technical problem to be solved by the present invention is to provide a nanocarrier and method of fabricating the same that may effectively deliver a biomaterial into a living plant-based object.

Furthermore, the technical problem to be solved by the present invention is to provide nanocarriers and methods of preparation fabricating the same that have good pH sensitivity and may have the property of releasing biomaterials within plant cells.

Furthermore, the technical problem to be solved by the present invention is to provide nanocarriers and methods of fabricating the same that are applicable regardless of plant species and that may reliably deliver appropriate biomaterials without limitation into a target plant species or an object related thereto.

Further, the technical problem to be solved by the present invention is to provide nanocarriers and methods of fabricating the same that may deliver biomaterials capable of promoting the regeneration of plant cells into a living plant-based object.

Furthermore, the technical problem to be solved by the present invention is to provide a nanocarrier-biomaterial complex comprising the aforementioned nanocarriers and a method of fabricating the same.

Furthermore, the technical problem to be solved by the present invention is to provide a method of treating an object using the aforementioned nanocarriers.

Furthermore, the technical problem to be solved by the present invention is to provide a method for promoting the regeneration of plant cells using the aforementioned nanocarriers.

The problems that the present invention is intended to solve are not limited to those mentioned above, and other problems not mentioned will be understood by those skilled in the art from the following description.

According to one embodiment of the present invention, there is provided a nanocarrier for delivering a biomaterial into a living plant-based object comprising: a nanotube; and a functional compound being bound to a surface of the nanotube by at least a polycyclic aromatic material and comprising an imidazole.

The polycyclic aromatic material may comprise a polycyclic aromatic hydrocarbon.

The polycyclic aromatic material may comprise a pyrenyl.

The nanocarrier may further comprise a maleimide bound to the polycyclic aromatic material and a cysteine bound to the functional compound, wherein the polycyclic aromatic material and the functional compound may be interconnected by bonding between the maleimide and the cysteine.

The functional compound may comprise a histidine comprising the imidazole.

The nanotubes may include carbon nanotubes.

The nanotubes may include single-walled carbon nanotubes (SWNTs).

The nanocarrier may comprise a Py-Hisₙ-SWNT structure, wherein the Py represents the pyrenyl, the His represents said the histidine comprising the imidazole, and the SWNTs may correspond to the nanotubes.

Where n may be greater than or equal to 2 and less than 100.

The object may comprise a callus.

The biomaterial may comprise ribonucleic acid (RNA).

According to another embodiment of the present invention, there is provided a nanocarrier for delivering a biomaterial into a living plant-based object comprising a nanotube; and a functional compound bound to a surface of the nanotube and including an imidazole, wherein the nanocarrier for delivering a biomaterial is configured to deliver a ribonucleic acid (RNA) that promotes a regeneration of plant cells in the object as the biomaterial into object.

According to another embodiment of the present invention, there is provided a nanocarrier-biomaterial complex for applying in a living plant-based object, the nanocarrier-biomaterial complex comprising: a nanocarrier of the foregoing; and a biomaterial bound to a functional compound of the nanocarrier.

The biomaterial may comprise ribonucleic acid (RNA).

An absolute value of a zeta potential of the nanocarrier-biomaterial complex may be greater than or equal to 20 mV.

A diameter of the nanocarrier-biomaterial complex is less than 10 nm.

The nanocarrier-biomaterial complex may have a property of releasing the biomaterial within plant cells of the object.

According to another embodiment of the present invention, there is provided a method of treating an object with a nanocarrier, comprising preparing a complex solution comprising the aforementioned nanocarrier-biomaterial complex; and delivering the nanocarrier-biomaterial complex into a living plant-based object by contacting the complex solution with the object.

The biomaterial may comprise a ribonucleic acid (RNA), wherein said RNA promotes a regeneration of plant cells of the object.

The object may comprise a plant tissue or a plant body.

The object may comprise a callus.

An absolute value of a zeta potential of the nanocarrier-biomaterial complex may be greater than or equal to 20 mV.

A diameter of the nanocarrier-biomaterial complex is less than 10 nm.

According to another embodiment of the invention, there is provided a method of treating an object with a nanocarrier comprising: preparing a complex solution including a nanocarrier-biomaterial complex which a nanocarrier comprising a functional compound comprising imidazole and a biomaterial comprising ribonucleic acid (RNA) are combined with the nanocarrier, delivering the nanocarrier-biomaterial complex into a living plant-based object by contacting the complex solution with the object; and promoting a regeneration of plant cells of the object using the RNA.

According to embodiments of the present invention, a nanocarrier may be implemented that may effectively deliver biomaterials into a living plant-based object. Furthermore, embodiments may implement nanocarriers that have good pH-sensitivity and may be characterized for releasing biomaterials within plant cells. Furthermore, embodiments may implement nanocarriers that are applicable regardless of plant species and may stably introduce a biomaterial suitable for a target plant species or an object associated therewith without limitation. Further, embodiments may implement nanocarriers capable of delivering biomaterials capable of promoting a regeneration of plant cells into a living plant-based object.

According to embodiments of the present invention, a nanocarrier-biomaterial complex comprising the above nanocarriers may be implemented. Further, embodiments provide methods of treating an object using the nanocarriers. Embodiments may also provide methods of promoting a regeneration of plant cells using the nanocarriers described above.

According to one embodiment, it is possible to implement a nanocarrier that may facilitate the delivery of external genetic material (biomaterial) that may be applied to improve the efficiency of regeneration of plant body, into plant cells. Furthermore, according to one embodiment the nanocarrier may be easy to apply to various plant species and provide a non-destructive and simple method of genetic material delivery, unlike gene delivery techniques using Agrobacterium tumefaciens or gene guns that have low delivery efficiency and require external physicochemical bombardment, which are limited in applicability to an available plant species.

Furthermore, according to one embodiment, unlike conventional carriers that have difficulty delivering genetic material such as RNA due to limitations in the shape or size of the biomaterial that may be bound, the nanocarrier with excellent pH sensitivity may be used to provide an efficient method of delivering biomaterial such as RNA into a living plant-based object such as callus. Technology according to one embodiment may effectively promote/promote plant regeneration by controlling a regeneration suppressor gene in callus.

However, the effects of the present invention are not limited to the above effects, and may be extended in various ways without departing from the technical ideas and scope of the present invention.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a drawing to illustrate a nanocarrier for delivering a biomaterial into a living plant-based object, in accordance with one embodiment of the present invention.

FIGS. 2A and 2B are drawings to exemplarily illustrate a method of fabricating a nanocarrier according to one embodiment of the present invention.

FIG. 3 is a drawing illustrating a nanocarrier-biomaterial complex for applying to a living plant-based object, in accordance with one embodiment of the present invention.

FIG. 4 is a diagram illustrating an exemplary method of treating an object using nanocarriers, in accordance with one embodiment of the present invention.

FIG. 5 is a photographic image showing a solution comprising Py-His₆-SWNTs synthesized according to one embodiment of the present invention.

FIG. 6 is a graph showing the results of measuring a zeta potential of Py-His₆-SWNTs synthesized according to one embodiment of the present invention.

FIG. 7 is a graph showing the results of measuring a size distribution of Py-His₆-SWNT particles synthesized according to one embodiment of the present invention.

FIG. 8 is a graph showing the variation of zeta potential with pH for Py-His₆-SWNTs synthesized according to one embodiment of the present invention and dCS^{cov}-SWNTs synthesized according to a comparative example.

FIG. 9 is a graph showing the results of evaluating the fluorescence properties of Cy3 before and after (GT)₍₁₅)-Cy3 is bound to Py-His₆-SWNTs synthesized according to one embodiment of the present invention.

FIG. 10 is a graph showing the results of evaluating the extent of biomaterial (ssDNA) release upon pH change of Py-His₆-SWNTs synthesized according to one embodiment of the present invention.

FIG. 11 is an image showing phenotypic observations of mitotic activity of callus introduced with Py-His₆-SWNTs synthesized according to embodiments of the present invention.

FIG. 12 is a photomicrographic image showing a callus treated with Py-His₆-SWNTs according to an embodiment of the present invention.

FIG. 13 is an image of confocal Raman microscopy analysis of the penetration characteristics of Py-His₆-SWNTs into callus treated with Py-His₍₆)-SWNTs by confocal Raman microscopy according to an embodiment of the present invention.

FIG. 14 is a graph showing the variation of zeta potential with mixing ratio of a complex comprising Py-His₆-SWNTs and RNA (STTM396) according to an embodiment of the present invention.

FIG. 15 is a graph showing the size variation with mixing ratio of a complex comprising Py-His₆-SWNTs and RNA (STTM396) according to an embodiment of the present invention.

FIG. 16 is a photomicrographic image showing callus treated with a complex of Py-His₆-SWNTs and RNA (STTM396) according to an embodiment of the present invention.

FIG. 17 is an image of confocal Raman microscopy analysis of the penetration characteristics of the complex into callus treated with a complex of Py-His₆-SWNTs and RNA (STTM396) according to an embodiment of the present invention.

FIG. 18 is an image illustrating a phenotypic evaluation of the regeneration of a callus after treating the callus according to embodiments and comparative examples of the present invention.

FIG. 19 is a graph showing the results of a quantitative evaluation of the regeneration of callus after treating the callus according to embodiments and comparative examples of the present invention.

In the following description, the same or similar elements are labeled with the same or similar reference numbers.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In addition, a term such as a “unit”, a “module”, a “block” or like, when used in the specification, represents a unit that processes at least one function or operation, and the unit or the like may be implemented by hardware or software or a combination of hardware and software.

Reference herein to a layer formed “on” a substrate or other layer refers to a layer formed directly on top of the substrate or other layer or to an intermediate layer or intermediate layers formed on the substrate or other layer. It will also be understood by those skilled in the art that structures or shapes that are “adjacent” to other structures or shapes may have portions that overlap or are disposed below the adjacent features.

In this specification, the relative terms, such as “below”, “above”, “upper”, “lower”, “horizontal”, and “vertical”, may be used to describe the relationship of one component, layer, or region to another component, layer, or region, as shown in the accompanying drawings. It is to be understood that these terms are intended to encompass not only the directions indicated in the figures, but also the other directions of the elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Preferred embodiments will now be described more fully hereinafter with reference to the accompanying drawings. However, they may be embodied in different forms and should not be construed as limited to the embodiments set forth herein. Rather, these embodiments are provided so that this disclosure will be thorough and complete, and will fully convey the scope of the disclosure to those skilled in the art.

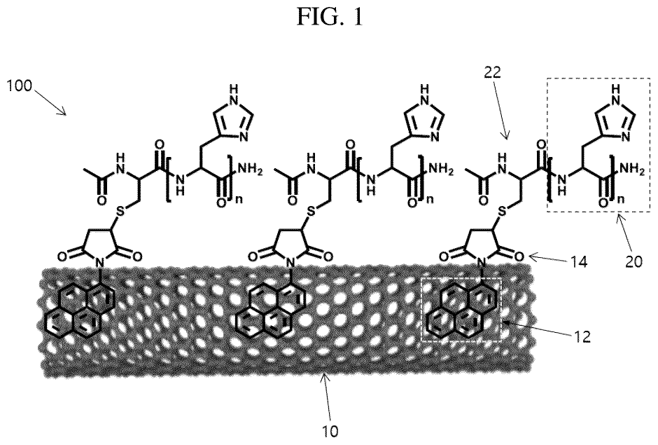

FIG. 1 is a drawing to illustrate a nanocarrier 100 for delivering a biomaterial into a living plant-based object, in accordance with one embodiment of the present invention.

Referring to FIG. 1, the nanocarrier 100 according to an embodiment of the present invention may comprise a nanotube 10 and a functional compound 20 that is bonded to a surface of the nanotube 10 and comprises an imidazole. The functional compound 20 may be bonded to the surface of the nanotube 10 by at least a polycyclic aromatic material. The polycyclic aromatic material may be or comprise, for example, a polycyclic aromatic hydrocarbon. The polycyclic aromatic material may be or comprise, for example, pyrenyl 12. The specific type of polycyclic aromatic material is not limited to pyrenyl 12 and may vary, as the case may be. In addition, according to other embodiments, the nanotubes 10 and the functional compound 20 may be interconnected by covalent bonds without an intermediate bonding material such as the polycyclic aromatic material. In addition, the manner of bonding between the nanotubes 10 and the functional compound 20 may be varied.

The nanotube 10 may be a carbon nanotube. For example, the nanotube 10 may be a single-walled carbon nanotube (SWNT). The length of the nanotube 10 may be from tens of nm to hundreds of nm, as a non-limiting example. The diameter of the nanotube 10 may be from about 1 nm to a few nm, as a non-limiting example. The diameter of the nanotubes 10 may be, for example, about 10 nm or less. The nanotube 10 may be a type of nanostructure. Further, the nanotube 10 may be a type of nanoparticle.

Carbon nanotubes may be useful biomaterial carriers to deliver biomaterials (e.g., genetic material) to plant tissues or inside plant bodies, beyond the limitations of conventional biomaterial carriers. Carbon nanotubes may be categorized into single-walled carbon nanotubes (SWNTs) and multi-walled carbon nanotubes (MWNTs) based on the number of walls. Carbon nanotubes have advantages such as high aspect ratio, stiffness, and large surface area, which may facilitate the delivery of biomaterials (biomolecules). In particular, SWNTs exhibit a characteristic Raman peak at about 1590 cm⁻¹, which may be used to observe the delivery characteristics of SWNTs into the target organism. However, despite their usefulness, carbon nanotubes have been limited in their use due to their low solubility and poor dispersion. In addition, in the case of cationic carbon nanotubes for gene carriers, there is a limit to the size or type of gene that may be delivered by allowing the gene to remain attached to the carbon nanotube (a type of nanoparticle) and induce the expression of its effect without falling off. In the case of SWNTs modified by positively charged SWNT modification methods (e.g., PEI-SWNTs, Chitosan-SWNTs), because the release of biomolecules from the SWNTs in the cell is limited, there are limits to the size and type of material that may be delivered.

However, in an embodiment of the present invention, the nanotubes 10 may be modified with a functional compound 20 comprising an imidazole to realize the property of the nanotubes 10 to inject into the cell with a biomaterial and then release the biomaterial within the cell. Here, the imidazole may be a type of a functional group. The nanotubes 10 may be modified with an imidazole or modified with a functional compound 20 comprising an imidazole. The imidazole may have a lower pKa value than the amine group. For example, the imidazole may have a pKa value of about 6.0. In this regard, the imidazole-modified nanotubes 10 may have a high pH sensitivity. Compared to positively charged SWNTs based on amine groups, imidazole-modified nanotubes 10 may have a higher pH sensitivity at neutral pH. Considering that the pH inside plant cells is on the order of 7˜8, it may be desirable to modify the nanotubes 10 with imidazole having a pKa of about 6.0. The imidazole-modified nanotubes 10 may have the property of delivering biomaterials (biomolecules) into the cell and then releasing the biomaterials more efficiently at a location with a neutral pH, such as the cytoplasm.

The functional compound 20 may be bonded to the surface of the nanotube 10 by at least a pyrenyl 12. The pyrenyl 12 may be bound to the surface of the nanotube 10 by a van der Waals force via a Π-Π interaction. The pyrenyl 12 may provide binding force to the functional compound 20 without damaging the properties of the nanotubes 10. Even though the pyrenyl 12 is bound to the surface of the nanotubes 10, characteristic Raman peaks due to the nanotubes 10 may appear.

According to one embodiment, the functional compound 20 may be a histidine comprising an imidazole or is a histidine itself. According to one example, the nanocarrier 100 may further comprise maleimide 14 bound to pyrenyl 12 and cysteine 22 bound to functional compound 20, wherein pyrenyl 12 and functional compound 20 may be mutually interconnected by the binding of maleimide 14 and cysteine 22. The maleimide 14 may be bound to the pyrenyl 12, the cysteine 22 may be bound to the maleimide 14, and the functional compound 20 may be coupled to the cysteine 22. Thus, the functional compound 20 may be bound to the nanotube 10 by the pyrenyl 12, maleimide 14, and cysteine 22. The maleimide 14 and cysteine 22 may provide good bonding properties between the pyrenyl 12 and the functional compound 20. Furthermore, the maleimide 14 and cysteine 22 may not interfere with the biomaterial transport properties and dispersibility of nanocarriers 100.

According to one embodiment, the nanocarrier 100 may comprise a Py-Hisₙ-SWNT structure. Here, the Py represents pyrenyl 12, the His represents a histidine comprising an imidazole, and the SWNT may correspond to the nanotube 10. Hisₙ may correspond to a functional compound (20). According to one example, n may be greater than or equal to 2 and less than 100. In one example, n may be greater than 2 but less than 100 or greater than 2 but less than 50. When n is 1, the binding properties of the biomaterial to the nanocarrier 100 may be reduced. On the other hand, if n is excessively large to a polymeric level, the dispersibility of the nanocarrier 100 may be reduced or the transport properties of the biomaterial may be reduced. According to an embodiment, by selecting an appropriate range of n, the binding properties of the biomaterial, the dispersibility of the nanocarrier 100, the transport properties of the biomaterial, and the like may be secured.

According to one embodiment, the living plant-based object may comprise plant tissue or a plant body. For example, the object may comprise a callus. A callus is a type of plant tissue, which may be an undifferentiated, undeformed mass of cells. Redifferentiation of plant cells from the callus may occur, resulting in the expression of a plant body. However, in embodiments of the present invention, the type of object is not limited to callus. In some cases, the object may be a plant organism, such as a plant seedling or a bud.

According to one embodiment, the biomaterial (or biological material) may be genetic material. The biomaterial may be a biomolecule. According to one example, the biomaterial may comprise a ribonucleic acid (RNA). The biomaterial may comprise synthetic RNA. In an example, the biomolecule may comprise a short tandem target mimic (STTM).

Complementary RNAs may be utilized to suppress the activity of miRNAs suppressing the regeneration of plant cells (tissues), and RNAs may be utilized to promote the regeneration of plant cells (tissues). However, according to an embodiment of the present invention, even biomaterials that are prone to degradation, such as RNA, may be easily and conveniently delivered into an object without additional chemical treatment or physical impact, and the efficiency of a regeneration of plant cells in the object may be improved. In this case, the nanocarrier 100 may be configured to deliver RNA as the biomaterial, which promotes the regeneration of plant cells in the object, into the object.

A short tandem target mimic (STTM) is a short single-stranded RNA created to artificially inhibit a small length RNA, such as a miRNA, and may be a nucleotide inhibitor that inhibits the function of the miRNA. STTMs may have complementary sequences for the target RNA to recognize a target at both ends, and have sequences in the middle to form a stem-loop for structural stability of the STTM inside the cell. The STTM may have specific and efficient inhibitory function on the target RNA, and may be used to inhibit the activity of small length RNAs. However, in embodiments of the present invention, the biomaterial is not limited to RNA/STTMs, and in some cases, the biomaterial may also comprise deoxyribonucleic acid (DNA). Furthermore, the biomaterial may comprise both RNA and DNA.

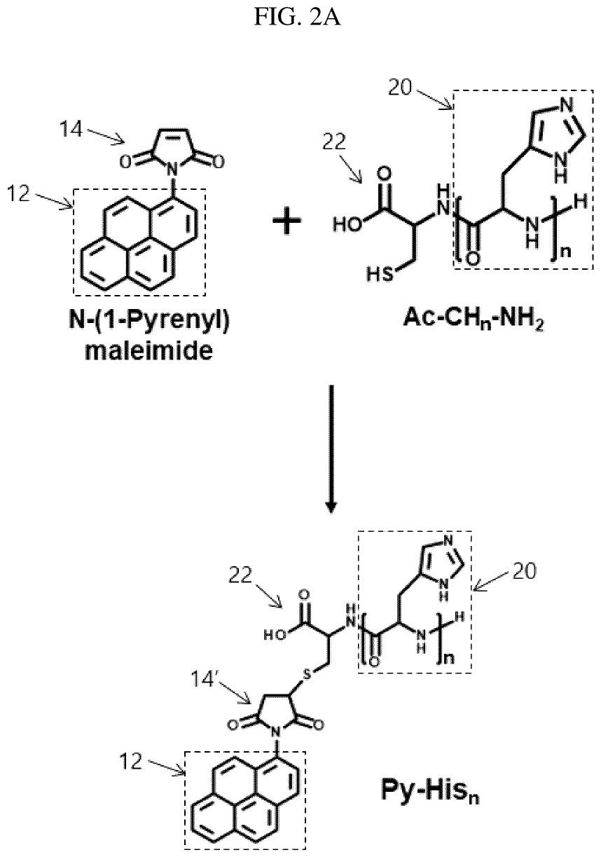

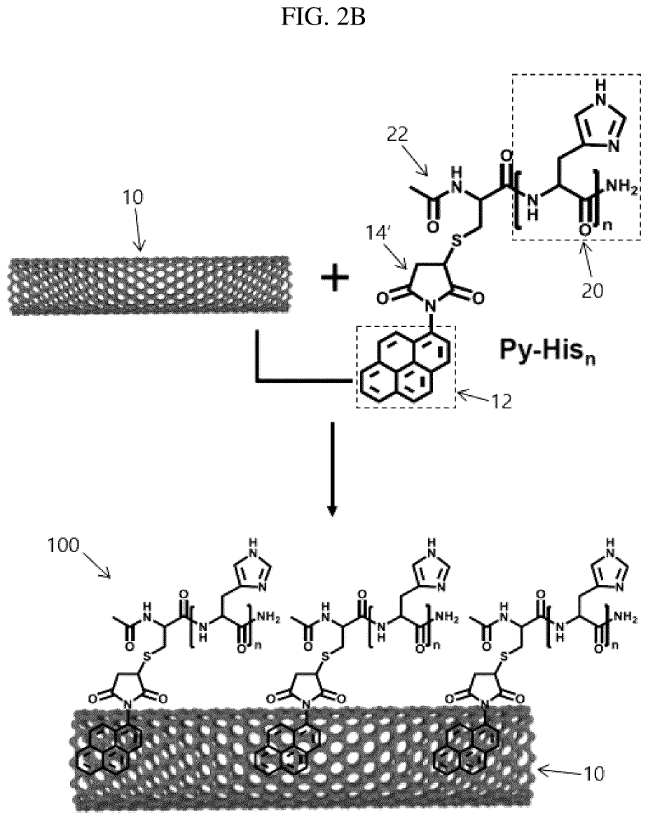

FIGS. 2A and 2B are drawings to exemplarily illustrate a method of fabricating a nanocarrier according to one embodiment of the present invention.

Referring to FIG. 2A, nanocarriers according to embodiments of the present invention may be prepared using a functional compound 20 comprising, for example, a histidine with imidazole. More specifically, an Ac—CHₙ—NH₂ with a cysteine 22 applied to at an end of n histidine may be prepared, which may then be reacted with pyrenyl maleimide, resulting in a Py-Hisₙ structure. Here, the Py represents pyrenyl 12, and the His represents histidine comprising the imidazole. The Hisₙ may correspond to a functional compound 20. In one example, n may be greater than or equal to 2 and less than 100. Pyrenyl 12 and functional compound 20 may be mutually bonded by the bonding of maleimide 14 and cysteine 22. Thiosuccinimide adduct (14′) may be formed by reacting of maleimide and thiol, in the Py-Hisₙ structure.

Referring to FIG. 2B, by inducing a Π-Π interaction between the pyrenyl 12 of Py-His₍ₙ₎ and the nanotube 10, the nanotube 10 modified with the functional compound 20 may be obtained. The nanocarrier 100 obtained in FIG. 2B may correspond to the nanocarrier 100 described in FIG. 1.

In one exemplary embodiment, a method of fabricating a nanocarrier may be as follows.

(1) Py-His₆ may be prepared by reacting 10 mg/ml of N-(1-pyrenyl) maleimide 150 μl and a peptide 150 μl consisting of 20 mg/ml of Ac—CH₆—NH₂ in a thermo mixer for 18 hours (1200 rpm, 25° C.)

(2) Add 2700 μl of distilled water to Py-His₆ to obtain 3 mL of diluted Py-His₆.

(3) A SWNT suspension may be prepared by placing 3 mL of 0.5 mg/ml of SWNTs 3 mL and Py-His₆ 3 mL in a container and performing tip sonication (1 hr, 40% amplitude, in an ice bath). A separation distance between the SWNTs is secured, allowing the pyrenyl to be better bound to the SWNTs

(4) To remove Py-His₆ that has not reacted (bound) with the SWNTs, the SWNT suspension may be dialyzed with distilled water for 48 hours (using a 100 kD dialysis membrane).

(5) The dialyzed solution is centrifuged at 17500 rpm for 4 hours, and about 80% of the supernatant is collected to obtain Py-His₆-SWNTs. The centrifugation may be used to remove large-sized nanostructures (nanoparticles).

However, the conditions and specific methods of fabricating the nanocarriers disclosed in the above experimental examples are exemplary only and may be varied.

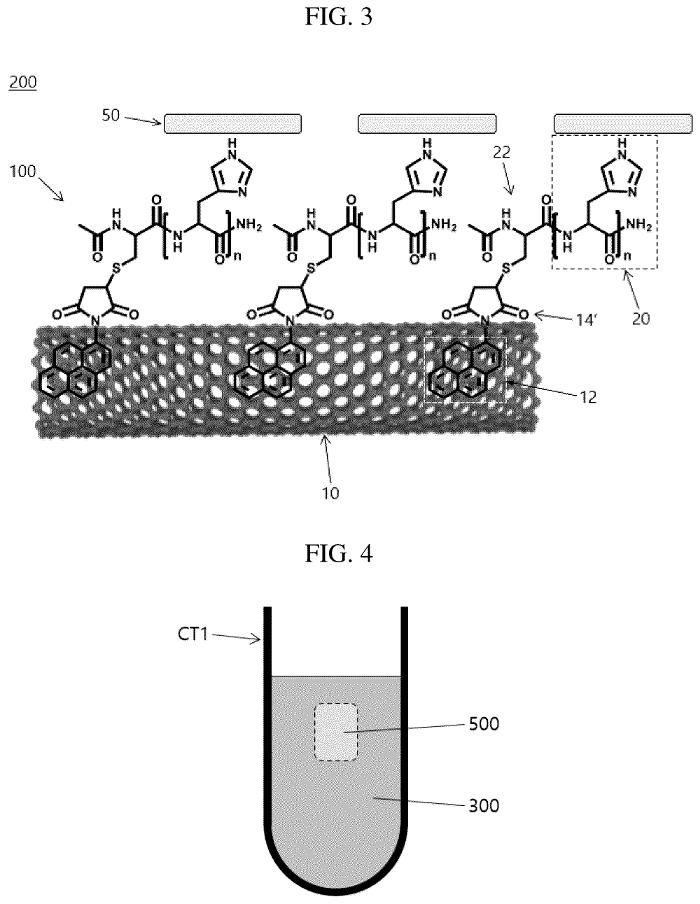

FIG. 3 is a diagram illustrating a nanocarrier-biomaterial complex 200 for applying to a living plant-based object, in accordance with one embodiment of the present invention.

Referring to FIG. 3, a nanocarrier-biomaterial complex 200 according to an embodiment of the present invention may comprise a nanocarrier 100 and a biomaterial 50 bound to a functional compound 20 of the nanocarrier 100. The nanocarrier 100 may correspond to the nanocarrier 100 according to the embodiment described with reference to FIG. 1. The biomaterial 50 may be coupled to the functional compound 20 by electrostatic interactions. The biomaterial 50 may comprise, for example, RNA. In this case, the biomaterial 50 may comprise a short tandem target mimic (STTM). RNAs that may inhibit the activity of miRNAs may be utilized to promote redifferentiation of plant cells (tissues). The nanocarrier 100 may be configured to deliver the RNA promoting a redifferentiation of plant cells in object as a biomaterial 50, into object. However, in embodiments of the present invention, the biomaterial 50 is not limited to RNA/STTM. In some cases, the biomaterial 50 may comprise DNA. Additionally, the biomaterial 50 may comprise both RNA and DNA.

According to one embodiment, a nanocarrier-biomaterial complex 200 may be fabricated by preparing a nanocarrier solution comprising the nanocarrier 100 and then mixing the biomaterial 50 into the nanocarrier solution. That is, a method of fabricating a nanocarrier-biomaterial complex according to an embodiment may include the steps of preparing a nanocarrier solution comprising the nanocarrier 100 and mixing the biomaterial 50 into the nanocarrier solution. The step of preparing the nanocarrier solution may include, for example, the method described with reference to FIGS. 2A and 2B.

In one embodiment, an absolute value of zeta potential of the nanocarrier-biomaterial complex 200 may be about 20 mV or greater. The diameter (outer diameter) of the nanocarrier-biomaterial complex 200 may be about 10 nm or less. If these conditions are met, the nanocarrier-biomaterial complex 200 may be well dispersed in solution and may penetrate into the cytosol. If the particle size of the nanocarrier-biomaterial complex 200 is too large or the particles are agglomerated, the dispersibility and penetration properties may be degraded. Furthermore, if the absolute value of the zeta potential of the nanocarrier-biomaterial complex 200 is greater than about 20 mV, the penetration properties into the cytoplasm may be improved.

According to one embodiment, the nanocarrier-biomaterial complex 200 may have the property of releasing the biomaterial 50 within a plant cell of the object. By including the functional compound 20 comprising imidazole, the nanocarrier 100 may have the property of injecting into the cell with the biomaterial 50 and then releasing the biomaterial 50 within the cell. The nanocarrier 100 comprising the imidazole may have a higher pH sensitivity at neutral pH. The nanocarrier 100 comprising the imidazole may have the property of delivering the biomaterial 50 into the cell and then releasing the biomaterial 50 more efficiently from a location having a neutral pH, such as the cytoplasm.

A method of treating an object with nanocarriers according to embodiments of the present invention may comprise the steps of preparing a complex solution comprising a nanocarrier-biomaterial complex and delivering the nanocarrier-biomaterial complex into a living plant-based object by contacting the complex solution with the object. The step of contacting the complex solution with the object may be a simple process of immersing or contacting the object in the complex solution. This may facilitate the delivery of a biomaterial loaded on the nanocarrier-biomaterial complex into the object. The biomaterial may comprise, for example, RNA, and the regeneration of plant cells of the object may be promoted by the RNA. The object may comprise plant tissue or a plant body. For example, the object may comprise a callus. The absolute value of the zeta potential of the nanocarrier-biomaterial complex may be about 20 mV or greater. The nanocarrier-biomaterial complex may have a diameter of about 10 nm or less. The nanocarrier-biomaterial complex may have the property of releasing the biomaterial within plant cells of the object. The complex solution may be an aqueous solution. Thus, for delivering the nanocarrier-biomaterial complex into the object, water may be used as a buffer.

According to one embodiment, a method of treating an object using a nanocarrier may comprise the steps of preparing a complex solution comprising a nanocarrier-biomaterial complex which a nanocarrier comprising a functional compound comprising an imidazole and a biomaterial comprising RNA are combined, and delivering the nanocarrier-biomaterial complex into a living plant-based object by contacting the complex solution with the object and promoting regeneration of plant cells of the object using the RNA.

Furthermore, any of the features described with reference to FIGS. 1 to 3 may be applied to a method of treating an object using nanocarriers according to embodiments of the present invention.

FIG. 4 is a diagram illustrating an exemplary method of treating an object using nanocarriers, in accordance with one embodiment of the present invention.

Referring to FIG. 4, a complex solution 300 comprising a nanocarrier-biomaterial complex may be prepared, and the complex solution 300 may be contacted with a living plant-based object 500 to deliver the nanocarrier-biomaterial complex into a living plant-based object 500 by contacting the complex solution 300 with the object 500. As a non-limiting example, the object 500 may be immersed or contacted with the complex solution 300 in a predetermined container CT1 containing the complex solution 300. The container CT1 may be of various types or sizes.

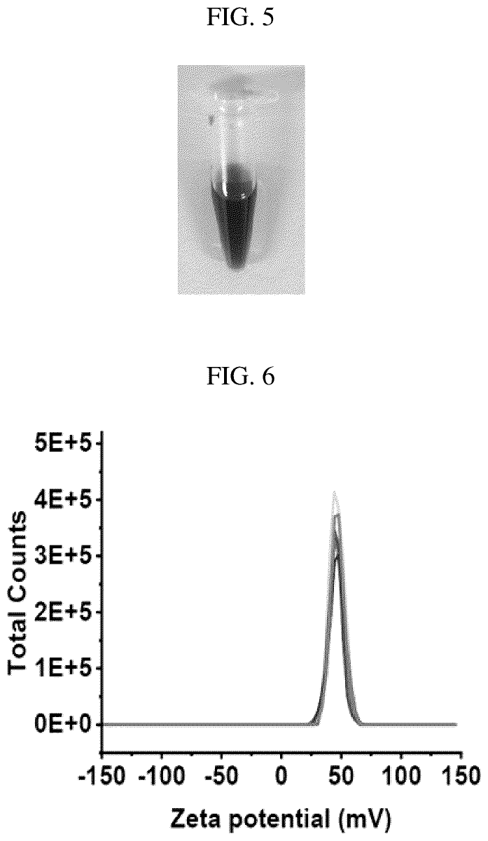

FIG. 5 is a photographic image showing a solution comprising Py-His₆-SWNTs synthesized according to one embodiment of the present invention. The solution may be a dispersion in which the Py-His₆-SWNT particles are dispersed.

FIG. 6 is a graph showing the results of measuring the zeta potential of Py-His₆-SWNTs synthesized according to one embodiment of the present invention. The zeta potential was obtained by dynamic light scattering (DLS) (Malvern Panalytical) measurements.

Referring to FIG. 6, it may be seen that the Py-His₆-SWNTs synthesized according to an embodiment have a positive zeta potential. Thus, Py-His₆-SWNTs as nanocarriers may have a positive charge and may be used as carriers of biomaterials with a negative charge. Biomaterials with negative charges may be well bound to Py-His₆-SWNTs.

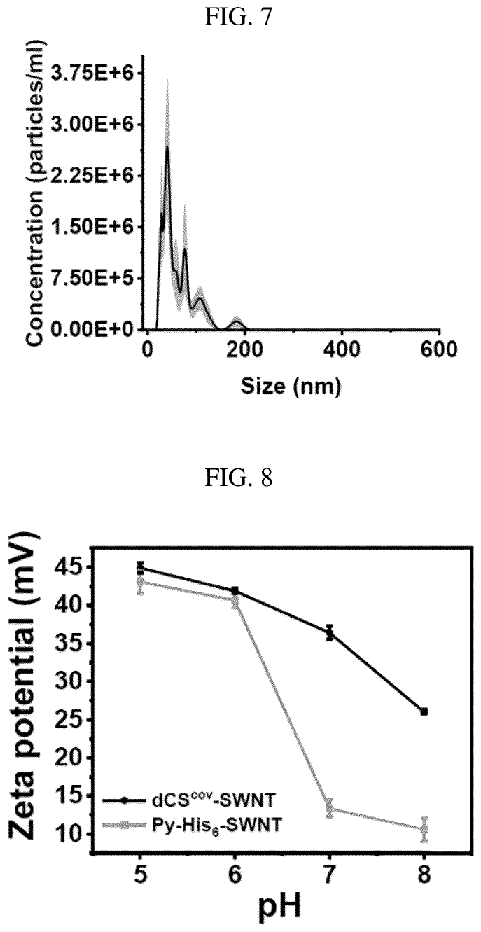

FIG. 7 is a graph showing the results of measuring the size distribution of Py-His₆-SWNT particles synthesized according to one embodiment of the present invention. The size distribution was obtained by Nanosight NM10 (Malvern Panalytical) measurements using nanoparticle tracking analysis (NTA).

Referring to FIG. 7, it may be seen that the synthesized Py-His₆-SWNT particles have a relatively small size and a uniform size distribution. The size measured in FIG. 7 is the hydrodynamic radius, which may be different from the actual size of the Py-His₆-SWNT particles.

FIG. 8 is a graph showing the change in zeta potential with pH for Py-His₆-SWNTs synthesized according to one embodiment of the present invention and dCS^{cov}-SWNTs synthesized according to a comparative example. Here, the dCS^{cov}-SWNTs according to the comparative example are Chitosan-SWNTs prepared by covalently attaching deacetylated chitosan to COOH-SWNTS.

Referring to FIG. 8, to confirm the pH sensitivity of Py-His₆-SWNTs, the zeta potential changes of Py-His₆-SWNTs in water with different pH (pH 5 to 8) were determined. For comparison, the pH sensitivity was compared with that of a conventional cationic polymer, Chitosan-SWNTs, i.e., dCS^{cov}-SWNTs with deacetylated chitosan covalently attached to COOH-SWNTs. As a result, we observed that Py-His₆-SWNTs exhibited faster charge neutralization with increasing pH compared to dCS^{cov}-SWNTs.

To determine the biomaterial release properties of nanocarriers inside plant cells according to embodiments of the present invention, experiments were performed using Cy3-conjugated (GT)₍₁₅₎. Cy3 is a fluorescent dye, and (GT)₁₅ is the full sequence name of ssDNA (single strand DNA).

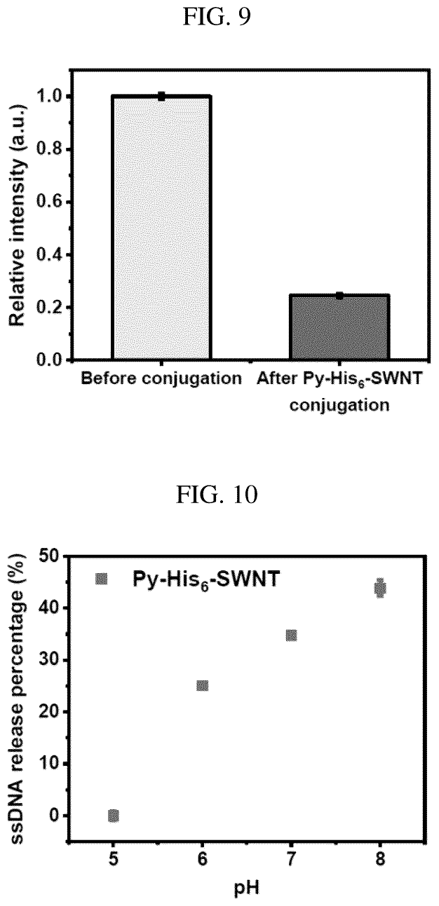

FIG. 9 is a graph showing the results of evaluating the fluorescence properties of Cy3 before and after (GT)₍₁₅)-Cy3 is bound to Py-His₆-SWNTs synthesized according to one embodiment of the present invention.

FIG. 10 is a graph showing the results of evaluating the extent of biomaterial (ssDNA) release upon pH change of Py-His₆-SWNTs synthesized according to one embodiment of the present invention.

As shown in FIG. 9, when (GT)₍₁₅₎-Cy3 is attached to a modified SWNT (i.e., Py-His₆-SWNT), the fluorescence of Cy3 is quenched and decreases in intensity compared to when it is alone, and the fluorescence intensity of Cy3 increases again when (GT)₁₅-Cy3 is moved away from the surface of the modified SWNT. Referring to FIG. 10, it may be seen that Py-His₆-SWNTs release a lot of ssDNA at a pH of about 7 to 8, which is the pH inside a plant cell.

The plant material (object) and its growth conditions used in embodiments of the present invention are described as follows.

In this embodiment of the invention, Columbia-0 ecotype Arabidopsis (Col-0) was used as the wild type. After sterilizing the surface of Arabidopsis seeds, they were planted and cultured on 0.7% agar plates containing ½ MS (Murashige and Skoog). Plant material was incubated under long-day conditions (16-h light/8-h dark cycle) using white fluorescent light (120 μmol photons per m⁻²·s⁻¹) at approximately 22 to 23° C. temperature. For plantlet callus formation, hypocotyl explants of 7-day-old plants were dark-cultured for 7 days at 22° C. in callus-inducing medium (MS medium supplemented with 0.5 μg/ml 2,4-dichlorophenoxyacetic acid and 0.05 μg/ml kinetin). However, these plant materials (objects) and growth conditions are exemplary only and may be varied.

The introduction method and analysis method of nanocarriers (a kind of nanoparticles) in callus used in embodiments of the present invention are described as follows.

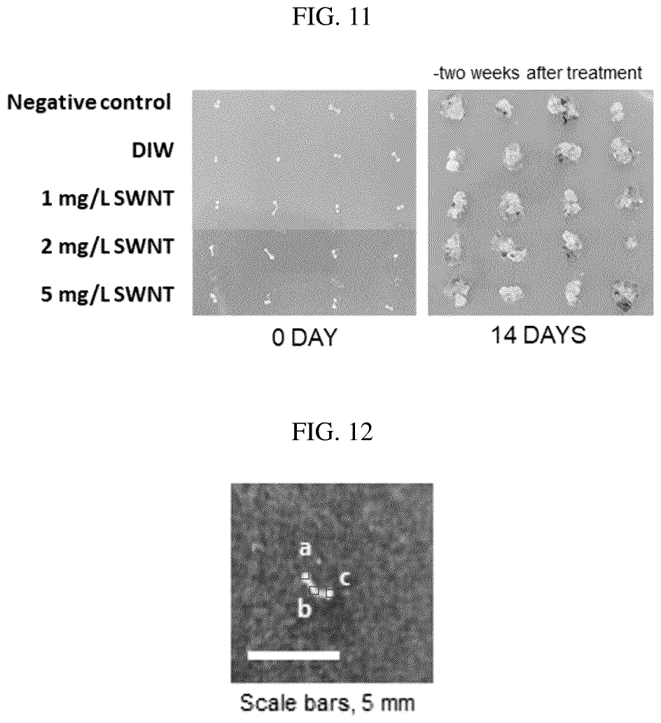

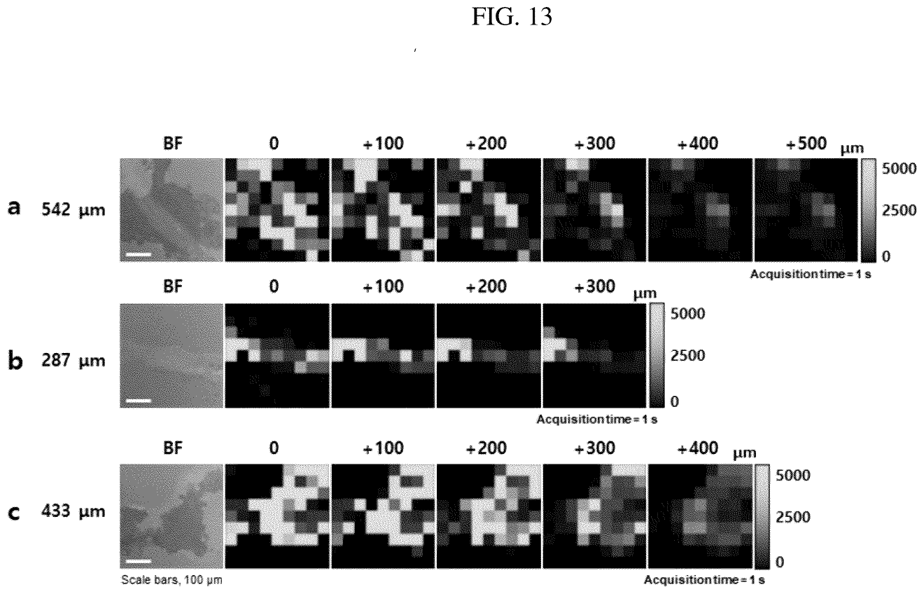

Callus was soaked in 200 μl of aqueous solutions of Py-His₆-SWNTs at various concentrations and incubated for 18 hr. The incubation was carried out in the dark, at room temperature. At the end of the incubation time, the callus was washed three times in 200 μl of distilled water to remove any remaining SWNTs, and the callus was then transferred to solid medium for observation. To determine the incubation time of Py-His₆-SWNTs on callus, callus activity after SWNT delivery was observed by phenotyping 14 days after completion of SWNT treatment (FIG. 11). Furthermore, the penetration efficiency of SWNTs into the callus interior was observed by Raman analysis by observing Raman bands at 1590 cm⁻¹, the characteristic Raman peak of SWNTs, both on the surface and inside the callus (FIG. 13). The results confirmed that under all Py-His₆-SWNT incubation conditions, Py-His₆-SWNTs had no effect (negative effect) on the fission activity of the callus, and the 18 h treatment of the callus with 2 mg/L Py-His₆-SWNTs (i.e., Py-His₆-SWNTs with a concentration of 2 mg/L of SWNT reference) resulted in the overall penetration of Py-His(6)-SWNTs inside the callus. However, the above-described method of introducing nanocarriers is exemplary only and may be varied.

FIG. 11 is an image showing the results of phenotypic observation of the fission activity of callus transfected with Py-His₆-SWNTs synthesized according to embodiments of the present invention. For comparison, FIG. 11 also includes results for callus treated with a negative control and deionized water (DIW).

Referring to FIG. 11, it may be seen that the division activity of the callus into which Py-His₆-SWNT was introduced, i.e., the callus treated with Py-His₆-SWNT, was not impaired. In all Py-His₆-SWNT incubation conditions, we found that Py-His₆-SWNT did not negatively affect the division activity of callus.

FIG. 12 is a photomicrographic image showing a callus treated with Py-His₆-SWNTs according to an embodiment of the present invention.

FIG. 13 is an image analyzed by confocal Raman microscopy characterizing the penetration of Py-His₆-SWNTs into callus treated with Py-His₆-SWNTs according to an embodiment of the present invention. a, b, and c in FIG. 13 are the results corresponding to points a, b, and c in FIG. 12. FIG. 13 is the result of an experiment using Py-His₆-SWNTs with 2 mg/L of SWNTs applied.

Referring to FIG. 13, the areas illustrated in grey gradients in the confocal Raman microscopy analysis images may represent areas where SWNTs are present. From these results, it may be seen that when the callus is treated with Py-His₆-SWNTs for a predetermined amount of time, the callus may be penetrated by Py-His₆-SWNTs throughout the callus interior.

An in vitro synthesis (i.e., in vitro transcription) method of STTM396 used in embodiments of the present invention is described below. Here, STTM396 is an example of a biomaterial used in an embodiment of the present invention.

In an embodiment of the present invention, for the in vitro synthesis of STTM396, which inhibits the activity of miRNA396, the template plasmid DNA into which the sequence of STTM396 (Sequence No. 1) was inserted was cloned using a two-fragment gibson assembly cloning method. Sequence analysis confirmed that the STTM396 sequence was that of the inserted template plasmid. The template plasmid DNA was linearized by cutting the 3′ end of the STTM396 sequence using Spe I restriction enzyme, and the template DNA was purified using the “Gel & PCR purification kit”. In vitro synthesis was performed using T7 RNA polymerase, followed by Dnase I treatment to remove the in vitro DNA. The synthesized STTM396 was purified using an RNA cleanup kit. However, the types of biomaterials and synthesis methods applicable to embodiments of the present invention are not limited to the foregoing.

In embodiments of the present invention, Py-His₆-SWNTs and RNA (i.e., STTM396) may be reacted at various ratios for about 10 minutes to form a complex of Py-His₆-SWNTs and RNA. The reaction ratio (mixing ratio) for the formation of the complex may be determined based on zeta potential and particle size data analyzed from dynamic light scattering (DLS) and nanoparticle tracking analysis (NTA) measurements.

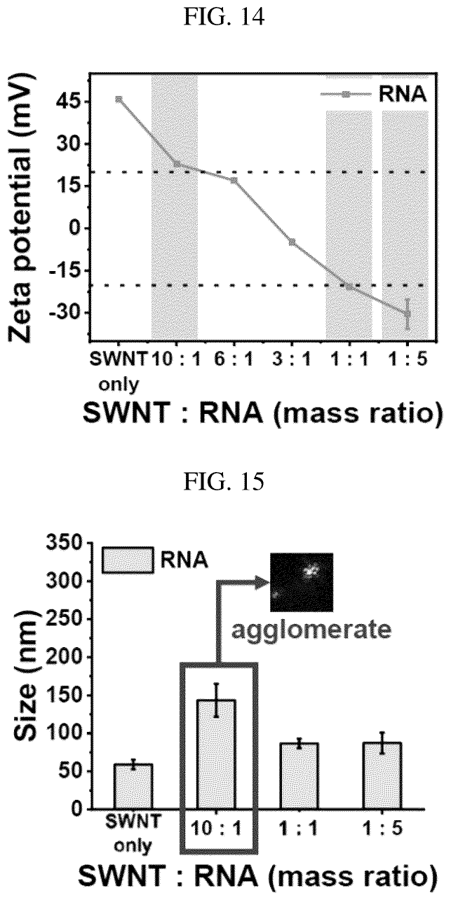

FIG. 14 is a graph showing the variation of the zeta potential as a function of the mixing ratio of a complex comprising Py-His₆-SWNTs and RNA (STTM396), according to an embodiment of the present invention. In FIG. 14, “SWNT only” refers to the presence of only Py-His₆-SWNT without the binding of RNA.

Referring to FIG. 14, the variation of the zeta potential of the complex as a function of the mixing ratio of Py-His₆-SWNTs and RNA (STTM396) may be seen. According to one embodiment, an absolute value of the zeta potential of the complex of Py-His₆-SWNT and RNA of about 20 mV or more may be advantageous for penetration into the cytoplasm of a subject. In this respect, for the embodiment of FIG. 14, a mixing ratio of Py-His₆-SWNTs and RNA of 10:1, 1:1, and 1:5 may be preferred. However, these results are exemplary only, and embodiments of the present invention are not limited thereto.

FIG. 15 is a graph showing the size variation as a function of mixing ratio of complexes containing Py-His₆-SWNTs and RNA (STTM396) according to embodiments of the present invention. In FIG. 15, “SWNT only” refers to the presence of Py-His₆-SWNTs alone without the binding of RNA.

Referring to FIG. 15, it may be seen that the size of the complex varies with the mixing ratio of Py-His₆-SWNT and RNA (STTM396). However, the size measured in FIG. 15 is the hydrodynamic radius, which may be different from the actual size of the complex. As a non-limiting example, the diameter (outer diameter) of the nanocarrier-biomaterial complex may preferably be about 10 nm or less.

In one embodiment, the absolute value of the zeta potential of the nanocarrier-biomaterial complex may be about 20 mV or more, and the diameter (outer diameter) of the nanocarrier-biomaterial complex may be about 10 nm or less. If these conditions are met, the nanocarrier-biomaterial complex may be well dispersed in solution and may penetrate into the cytosol.

The following describes a method for introducing a complex of Py-His₆-SWNT and RNA (STTM396) in callus used in embodiments of the present invention and a method for analyzing the introduced complex.

The above complex of Py-His₆-SWNTs and RNA (STTM396) may be dark-incubated into callus at 2 mg/L of SWNTs for 18 hours. The transfection of the complex of Py-His₍₆₎-SWNT and RNA into callus was performed as described previously. The penetration characteristics/efficiency of the complexes into callus were then observed by Raman analysis.

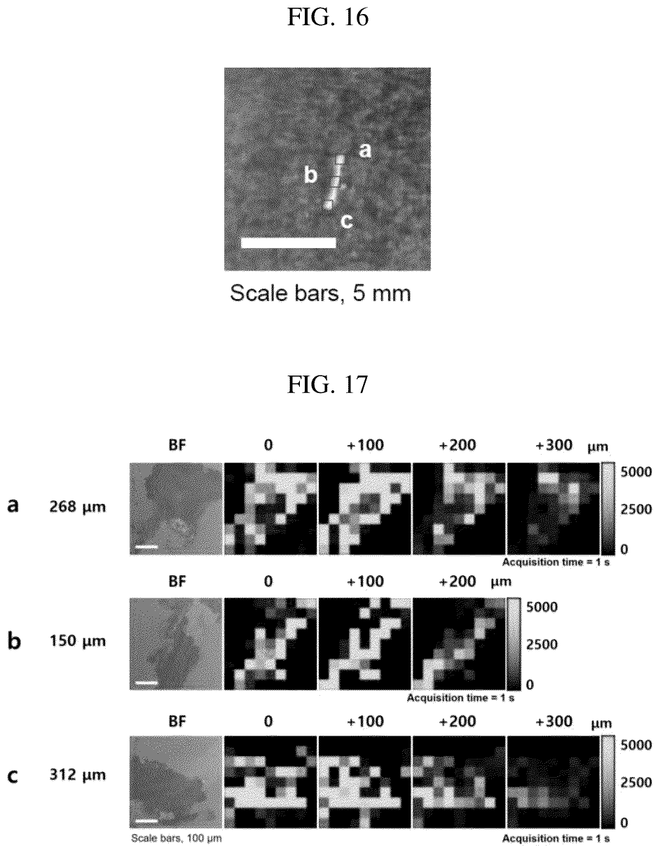

FIG. 16 is a photomicrographic image showing callus treated with a complex of Py-His₆-SWNTs and RNA (STTM396) according to an embodiment of the present invention.

FIG. 17 is an image of confocal Raman microscopy analysis of the penetration characteristics of the complex into callus treated with a complex of Py-His₆-SWNTs and RNA (STTM396) according to an embodiment of the present invention. Points a, b, and c in FIG. 17 correspond to points a, b, and c in FIG. 16. FIG. 17 is the result of an experiment using Py-His₆-SWNTs with 2 mg/L of SWNTs applied. Also, in FIG. 17, the mass ratio of Py-His₆-SWNTs to RNA was 1:1.

Referring to FIG. 17, the areas illustrated in grey gradients in the confocal Raman microscopy analysis images may represent the areas where SWNTs are present. From these results, it may be seen that when callus was treated with the complex of Py-His₆-SWNTs and RNA for a predetermined amount of time, the complex was able to penetrate the entire surface and interior of the callus.

Techniques according to embodiments of the present invention may provide conditions for complex formation where the nanocarriers do not lose their dispersibility after being loaded with biomaterials (e.g., RNA), and may provide culture conditions where the complexes may be loaded into callus to effectively deliver biomaterials without losing callus activity. Furthermore, the complexes according to embodiments of the present invention may have a pH-dependent release of biomaterials, which may facilitate the delivery of biomaterials into plant cells. Thus, the technology according to embodiments may be utilized as a technique for selective gene expression control at specific times during the callus tissue culture process.

Embodiments of the present invention may be used to deliver biomaterials to plant tissues or plant bodies through the simple process of mixing biomaterials with imidazole-modified SWNTs to form a complex, immersing the plant in a solution containing the complex, and delivering the biomaterials to the plant tissue or plant body without any physicochemical impact. Embodiments of the present invention may be applicable to neo-sarcoma development techniques, plant biotechnology-based pharmaceutical production, and the like.

The method for redifferentiation phenotyping of callus treated with a complex of Py-His₆-SWNT and RNA (STTM396) used in embodiments of the present invention is as follows.

The complex of Py-His₆-SWNTs with RNA (STTM396) in a 1:1 mass ratio was used to treat callus at concentrations of 2 mg/L and 5 mg/L based on SWNTs for 18 hours under dark conditions. The introduction of Py-His₆-SWNTs and RNA complexes into callus was the same as previously described. For callus redifferentiation, the above-treated callus was cultured in shoot-inducing medium (MS medium supplemented with 0.9 μmol/L 3-indoleacetic acid, 2.5 μmol/L 2-isopentenyladenine) at 25° C., under continuous light conditions for 21 days. The results showed that treatment of callus with the complex of Py-His₆-SWNTs and RNA increased the cellular redifferentiation rate of callus. However, the method of cell differentiation assay using the complex is not limited to the above and may be varied.

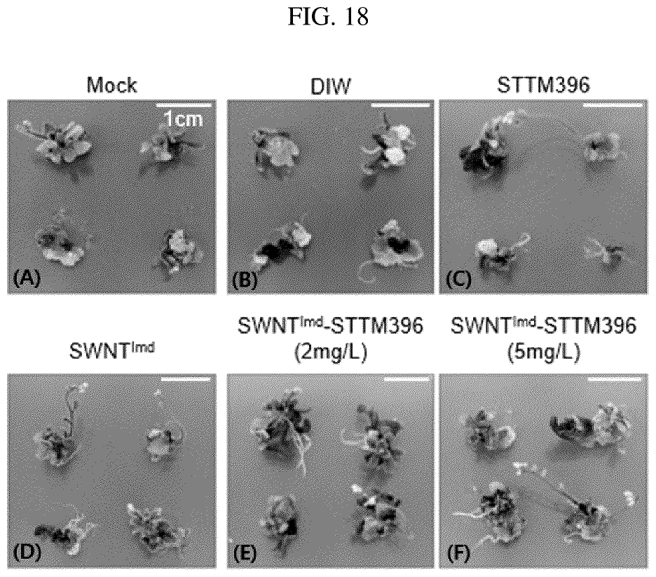

FIG. 18 is an image illustrating a phenotypic evaluation of the redifferentiation of a callus after processing the callus according to embodiments and comparative examples of the present invention.

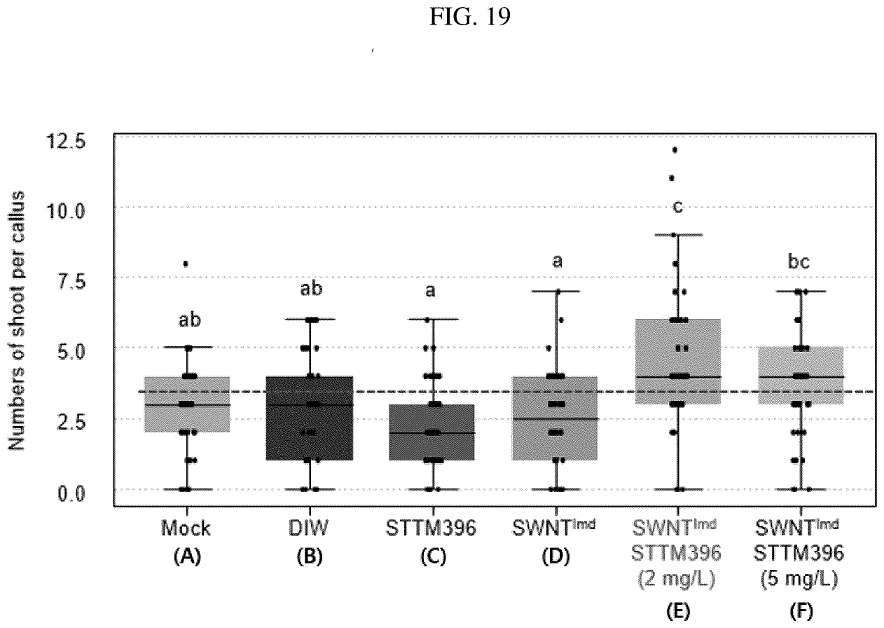

FIG. 19 is a graph showing the results of a quantitative evaluation of the re-differentiation of callus after treating the callus according to embodiments and comparative examples of the present invention.

In FIGS. 18 and 19, (A) through (D) correspond to comparative examples, and (E) and (F) correspond to embodiments. (A) corresponds to the mock case as a control. (B) is the case where the callus was treated with DIW. (C) Treatment of callus with STTM396. (D) Treatment of callus with imidazole-modified SWNTs (i.e. Py-Hisₙ-SWNTs). (E) and (F) are treatment of callus with a complex of Py-His₆-SWNTs and RNA (STTM396) according to an embodiment. (E) Callus was treated with SWNTs at a concentration of 2 mg/L, and (F) Callus was treated with SWNTs at a concentration of 5 mg/L.

Referring to FIGS. 18 and 19, it may be seen that treatment of callus with the complex of Py-His₆-SWNT and RNA (STTM396) resulted in an increased rate of cellular redifferentiation of callus in the embodiments compared to the comparative examples.

According to embodiments of the invention described above, it is possible to implement nanocarriers that may effectively deliver biomaterials into a living plant-based object. Furthermore, embodiments may implement nanocarriers that have good pH sensitivity and may be characterized for releasing biomaterials within plant cells. Furthermore, embodiments may implement nanocarriers that are applicable regardless of plant species and may reliably deliver appropriate biomaterials without limitation into a target plant species or an object related thereto. Further, embodiments may implement nanocarriers capable of delivering biomaterials capable of promoting the redifferentiation of plant cells into a living plant-based object.

According to embodiments of the present invention, nanocarrier-biomaterial complexes comprising the above nanocarriers may be implemented. Further, embodiments provide methods of treating an object using the nanocarriers. Embodiments may also provide methods of promoting redifferentiation of plant cells using the nanocarriers described above.

According to one embodiment, it is possible to implement a nanocarrier that may facilitate the delivery of external genetic material (biomaterial) into plant cells that may be applied to improve the efficiency of redifferentiation of plant organisms. Furthermore, according to one embodiment the nanocarrier may be easy to apply to various plant species and provide a non-destructive and simple method of genetic material delivery, unlike gene delivery techniques using Agrobacterium tumefaciens or gene guns that have low delivery efficiency and require external physicochemical bombardment, which are limited in applicability to an available plant species.

Furthermore, according to one embodiment, unlike conventional carriers that have difficulty delivering genetic material such as RNA due to limitations in the shape or size of the biomaterial that may be bound, the nanocarrier with excellent pH sensitivity may be used to provide an efficient method of delivering biomaterial such as RNA into a living plant-based object such as callus. Technology according to one embodiment may effectively promote/promote plant regeneration by controlling a regeneration suppressor gene in callus.

This description discloses preferred embodiments of the present invention, and although certain terms are used, they are used in a general sense only to facilitate the description and understanding of the invention and are not intended to limit the scope of the invention. In addition to the embodiments disclosed herein, other modifications based on the technical ideas of the present invention will be apparent to those of ordinary skill in the art to which the present invention belongs. It will be apparent to one of ordinary skill in the art that the nanocarriers for biomaterial delivery, nanocarrier-biomaterial composites, methods of preparation thereof, and methods of treating living plant-based objects with nanocarriers, according to the embodiments described with reference to FIGS. 1 to 19, may be subject to various substitutions, modifications, and variations without departing from the technical ideas of the invention. The scope of the invention is therefore not to be defined by the embodiments described, but by the technical ideas recited in the patent claims.

While the present disclosure has been described with reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims.

The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. A nanocarrier for delivering biomaterials into a living plant-based object, the nanocarrier comprising:
a nanotube; and
a functional compound bound to a surface of the nanotube by at least a polycyclic aromatic material and including an imidazole.

2. The nanocarrier of claim 1, wherein the polycyclic aromatic material comprising a pyrenyl.

3. The nanocarrier of claim 1, further comprising a maleimide bound to the polycyclic aromatic material and a cysteine bound to the functional compound,
wherein the polycyclic aromatic material and the functional compound are interconnected by bonding between the maleimide and the cysteine.

4. The nanocarrier of claim 1, wherein the functional compound comprises a histidine including the imidazole.

5. The nanocarrier of claim 1, wherein the nanotube comprises a carbon nanotube.

6. The nanocarrier of claim 5 wherein the nanotube comprises a single-walled carbon nanotube (SWNT).

7. The nanocarrier of claim 2, the nanocarrier comprises a Py-Hisₙ-SWNT structure,
where the Py represents the pyrenyl, the His represents the histidine comprising the imidazole, and the SWNT corresponds to the nanotube.

8. The nanocarrier of claim 7, wherein the n is greater than or equal to 2 and less than 100.

9. The nanocarrier of claim 1, the object comprises a callus.

10. The nanocarrier of claim 1, the biomaterial comprises a ribonucleic acid (RNA).

11. A nanocarrier for delivering biomaterials into a living plant-based object, the nanocarrier comprising:
a nanotube; and
a functional compound bound to a surface of the nanotubes and including an imidazole,
wherein the nanocarrier is configured to deliver a ribonucleic acid (RNA) that promotes a regeneration of plant cells of the object as the biomaterial, into the object.

12. A nanocarrier-biomaterial complexes for applying in living plant-based object, the nanocarrier-biomaterial complexes comprising:
the nanocarrier of claim 1; and
a biomaterial bound to the functional compound of the nanocarrier.

13. The nanocarrier-biomaterial complexes of claim 12, wherein the biomaterial comprises a ribonucleic acid (RNA).

14. The nanocarrier-biomaterial complexes of claim 12,
wherein an absolute value of a zeta potential of the nanocarrier-biomaterial complex is greater than or equal to 20 mV,
wherein a diameter of the nanocarrier-biomaterial complex is less than 10 nm.

15. The nanocarrier-biomaterial complexes of claim 12, wherein the nanocarrier-biomaterial complex has a property of releasing the biomaterial within a plant cell of the object.

16. A method of treating an object with a nanocarrier comprising:
preparing a complex solution including the nanocarrier-biomaterial complex of claim 12; and
delivering the nanocarrier-biomaterial complex into a living plant-based object by contacting the complex solution with the object.

17. The method of claim 16,
wherein the biomaterial comprises a ribonucleic acid (RNA),
wherein the RNA promotes a regeneration of plant cells of the object.

18. The method of claim 16, wherein the object comprising a plant tissue, a plant body or a callus.

19. The method of claim 16,
wherein an absolute value of a zeta potential of the nanocarrier-biomaterial complex is greater than or equal to 20 mV,
wherein a diameter of the nanocarrier-biomaterial complex is less than 10 nm.

20. A method of treating an object with a nanocarrier comprising:
preparing a complex solution including a nanocarrier-biomaterial complex which a nanocarrier comprising a functional compound comprising imidazole and a biomaterial comprising ribonucleic acid (RNA) are combined;
delivering the nanocarrier-biomaterial complex into a living plant-based object by contacting the complex solution with the object; and
promoting a regeneration of plant cells of the object using the RNA.
